(19) United States
(12) Patent Application Publication
(10) Pub. No.: US 2024/0094173 A1
(43) Pub. Date: Mar. 21, 2024

(54) A METHOD OF ANALYSIS OF MASS SPECTROMETRY DATA

(71) Applicants: Micromass UK Limited, Wilmslow (GB); Waters Technologies Ireland Limited, Dublin (IE)

(72) Inventors: Patrick Boyce, Wilmslow (GB); Keith Richardson, Wilmslow (GB)

(21) Appl. No.: 18/264,217

(22) PCT Filed: Feb. 2, 2022
(86) PCT No.: PCT/GB2022/050278

§ 371 Date: Aug. 3, 2023

(30) Foreign Application Priority Data
Feb. 3, 2021 (GB) 2101497.2
Sep. 30, 2021 (GB) 2114010.8

(51) Int. Cl. G01N 30/86

(52) U.S. Cl. CPC G01N 30/8631; G01N 30/8679; G01N 30/8696; G01N 2030/8648

(57) ABSTRACT

There is provided a method of analysis of mass spectrometry data comprising obtaining raw experimental mass spectrometry data; performing a first deconvolution of the raw experimental mass spectrometry data using a deconvolution algorithm, a wide first input parameter set, and a wide first output parameter set to obtain a deconvolved output; obtaining discrete peak data from the deconvolved output; simulating raw data for a first peak of the discrete peak data to obtain reference simulated raw discrete data; simulating raw data for a second peak of the discrete peak data to obtain suspect simulated raw discrete data; and determining whether the second peak is likely an artefact or indicative of a mass by comparing the suspect simulated raw discrete data with the reference simulated raw discrete data.

### FIELD

The present disclosure relates to a method of analysis of mass spectrometry data, specifically, although not exclusively, to a method of determining whether a peak in a deconvolved output is likely an artefact or indicative of a mass.

### BACKGROUND

Deconvolution of raw experimental mass spectra using deconvolution algorithms is known. In the deconvolution of raw experimental mass spectra, raw experimental mass spectrometry data, typically a plot of m/z against intensity, is deconvolved to provide a deconvolved output, typically a plot of mass against intensity. There are a number of known algorithms for deconvolution of raw experimental mass spectrometry data to provide deconvolved outputs. Deconvolution algorithms may produce artefacts in deconvolved outputs, particularly when non-ideal settings are used to decrease processing time or because of uncertainty in the content of the data. A typical artefact is a peak in the deconvolved output of mass against intensity indicative of a mass of an analyte, when the analyte is not present in the sample analysed.

It is a non-exclusive object of the present disclosure to address the problem of artefacts in deconvolved outputs.

### BRIEF DESCRIPTION OF THE INVENTION

There is provided a method of analysis of mass spectrometry data comprising:
obtaining raw experimental mass spectrometry data;
performing a first deconvolution of the raw experimental mass spectrometry data using a deconvolution algorithm, a wide first input parameter set, and a wide first output parameter set to obtain a deconvolved output;
obtaining discrete peak data from the deconvolved output;
simulating raw data for a first peak of the discrete peak data to obtain reference simulated raw discrete data;
simulating raw data for a second peak of the discrete peak data to obtain suspect simulated raw discrete data; and
determining whether the second peak is likely an artefact or indicative of a mass by comparing the suspect simulated raw discrete data with the reference simulated raw discrete data.

The first peak of the discrete peak data may be the most intense peak of the discrete peak data.

The second peak of the discrete peak data may be the closest mass to the first peak of the discrete peak data.

Comparing the suspect simulated raw discrete data with the reference simulated raw discrete data may comprise comparing the m/z values of the suspect simulated raw discrete data with the m/z values of the reference simulated raw discrete data.

The comparing the m/z values of the suspect simulated raw discrete data with the m/z values of the reference simulated raw discrete data may comprise calculating the width of the theoretical isotope distribution at the charge state z of the m/z value under consideration.

The second peak may be identified as likely an artefact if all of the m/z values of the suspect simulated raw discrete data are within the m/z values of the reference simulated raw discrete data.

The second peak may be identified as likely indicative of a mass if an m/z value of the suspect simulated raw discrete data is not within the m/z values of the reference simulated raw discrete data.

Once the second peak is identified as likely indicative of a mass, comparing the suspect simulated raw discrete data with the reference simulated raw discrete data may be ceased.

Once the second peak is identified as likely indicative of a mass, the suspect simulated raw discrete data may be added to the reference simulated raw discrete data.

The method of analysis of mass spectrometry data may
further comprise simulating raw data for a further peak of the discrete peak data to obtain further suspect simulated raw discrete data; and
further comprise determining whether the further peak is likely an artefact or indicative of a mass by comparing the further suspect simulated raw discrete data with the reference simulated raw discrete data.

The method of analysis of mass spectrometry data may further comprise:
determining a narrow second input parameter set, comprising:
setting an input spectrum threshold percentage;
setting the smallest m/z value in the reference simulated raw discrete data above the input spectrum threshold percentage as a lower bound of the narrow second input parameter set; and/or
setting the largest m/z value in the reference simulated raw discrete data above the input spectrum threshold percentage as an upper bound of the narrow second input parameter set.

Determining the narrow second input parameter set may further comprise:
if the second peak is determined as likely indicative of a mass:
and if the smallest m/z value in the suspect simulated raw discrete data above the input spectrum threshold percentage is smaller than the lower bound of the narrow second input parameter set, setting the smallest m/z value in the suspect simulated raw discrete data as the lower bound of the narrow second input parameter set; and/or
and if the largest m/z value in the suspect simulated raw discrete data above the input spectrum threshold percentage is greater than the upper bound of the narrow second input parameter set, setting the largest m/z value in the suspect simulated raw discrete data as the upper bound of the narrow second input parameter set.

Determining the narrow second input parameter set may further comprise:
if the or a further peak is determined as likely indicative of a mass:
if the smallest m/z value in the further suspect simulated raw discrete data above the input spectrum threshold percentage is smaller than the lower bound of the narrow second input parameter set, setting the smallest m/z value in the further suspect simulated raw discrete data as the lower bound of the narrow second input parameter set; and/or
if the largest m/z value in the further suspect simulated raw discrete data above the input spectrum threshold percentage is greater than the upper bound of the narrow second input parameter set, setting the largest m/z value in the suspect simulated raw discrete data as the upper bound of the narrow second input parameter set.

The input spectrum threshold percentage may be set to zero.

The method of analysis of mass spectrometry data may further comprise:
determining a narrow second output parameter set, comprising:
setting an offset value; and
setting the smallest of the first peak, the second peak, and/or any further peak(s) determined to be indicative of a mass minus the offset value as a lower bound of the narrow second output parameter set; and/or
setting the largest of the first peak, the second peak, and/or any further peak(s) determined to be indicative of a mass plus the offset value as an upper bound of the narrow second output parameter set.

The method of analysis of mass spectrometry data may further comprise:
determining a narrow second output parameter set, comprising:
setting an offset value; and
setting the first peak, the second peak, and/or any further peak(s) determined to be indicative of a mass plus and minus the offset value as included within the narrow second output parameter set.

The method of analysis of mass spectrometry data may further comprise:
performing a second deconvolution of the raw experimental mass spectrometry data using a deconvolution algorithm and the narrow second input parameter set determined using a method herein; and/or the narrow second output parameter set determined using a method herein to obtain a second deconvolved output.

There is also provided method of determining whether a second peak in a deconvolved output is likely an artefact or indicative of a mass comprising:
obtaining discrete peak data from the deconvolved output;
simulating raw data for a first peak of the discrete peak data to obtain reference simulated raw discrete data;
simulating raw data for the second peak of the discrete peak data to obtain suspect simulated raw discrete data; and
determining whether the second peak is likely an artefact or indicative of a mass by comparing the suspect simulated raw discrete data with the reference simulated raw discrete data.

The method of determining whether a second peak in a deconvolved output is likely an artefact or indicative of a mass may further comprise one or more or all of the features of the method of analysis of mass spectrometry data described herein.

There is also provided a method of analysis of mass spectrometry data comprising:
obtaining raw experimental mass spectrometry data;
performing a first deconvolution of the raw experimental mass spectrometry data using a deconvolution algorithm, a wide first input parameter set, and a wide first output parameter set to obtain a deconvolved output;
identifying peaks in the deconvolved output;
simulating raw data for a first peak of the deconvolved output to obtain reference simulated raw data;
simulating raw data for a second peak of the deconvolved output to obtain suspect simulated raw data;
determining a co-efficient of overlap between the suspect simulated raw data and the reference simulated raw data; and
determining whether the second peak is likely an artefact or indicative of a mass by comparing the co-efficient of overlap to a predetermined threshold.

The determining a co-efficient of overlap between the suspect simulated raw data and the reference simulated raw data may comprise determining the co-efficient of overlap for a peak or collection of peaks in the suspect simulated raw data with a peak or collection of peaks in the reference simulated raw data.

The determining a co-efficient of overlap between the suspect simulated raw data and the reference simulated raw data may comprise determining the co-efficient of overlap for all or substantially all of the suspect simulated raw data and of the reference simulated raw data.

There is also provided a method of determining whether a second peak in a deconvolved output is likely an artefact or indicative of a mass comprising:
identifying peaks in the deconvolved output;
simulating raw data for a first peak of the deconvolved output to obtain reference simulated raw data;
simulating raw data for a second peak of the deconvolved output to obtain suspect simulated raw data;
determining a co-efficient of overlap between the suspect simulated raw data and the reference simulated raw data; and
determining whether the second peak is likely an artefact or indicative of a mass by comparing the co-efficient of overlap to a predetermined threshold.

The method of determining whether a second peak in a deconvolved output is likely an artefact or indicative of a mass may further comprise one or more or all of the features of the method of analysis of mass spectrometry data described herein.

There is also provided a computer readable medium having instructions stored thereon which, when executed by a processor, cause the performance of a method described herein.

There is also provided a computer program including instructions which, when executed by a processor, cause the performance of a method described herein.

There is also provided a system including at least one processor and a computer readable medium, wherein the computer readable medium has instructions stored thereon which, when executed by the at least one processor, cause the system to perform a method described herein.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a flow diagram of a method in accordance with an embodiment of the present disclosure;

FIG. 2 shows an example discrete peak data from a deconvolved output for use in accordance with an embodiment of the present disclosure;

FIG. 3 shows an example of reference simulated raw discrete data obtained from a first peak of the discrete peak data of FIG. 2;

FIG. 4 shows an example of suspect simulated raw discrete data obtained from a second peak of the discrete peak data of FIG. 2;

FIG. 5 shows the reference simulated raw discrete data of FIG. 3 (top) adjacent the suspect simulated raw discrete data of FIG. 4 (bottom);

FIG. 6 shows an example reference simulated raw discrete data for use in determining a narrow second input parameter set in accordance with an embodiment of the present disclosure; and

FIG. 7 shows an example second deconvolved output obtained in accordance with an embodiment of the present disclosure.

### DETAILED DESCRIPTION OF THE DISCLOSURE

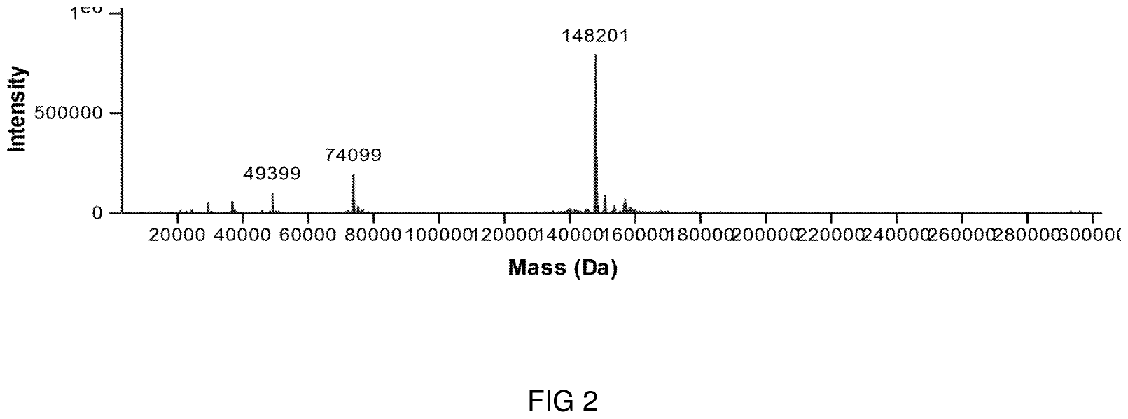

From viewing raw experimental mass spectrometry data it is not immediately apparent how many analytes are present in a sample analysed or what the mass(es) of the analyte(s) present in the sample analysed was/were. In particular, some ionisation methods (for example electrospray) produce multiple charge states, and hence multiple mass spectral peaks, for each analyte. Peaks from different analytes can overlap in mass-to-charge ratio and can also lie on top of each other. Therefore, in order to try and determine the mass(es) of analyte(s) in a sample an operator might utilise a deconvolution algorithm. A deconvolution algorithm might, for example, make use of Maximum Entropy or Bayesian or least-squares based techniques. An output of a deconvolution algorithm is shown in FIG. 2.

When utilising a deconvolution algorithm it is common to specify an expected output mass range, i.e. an output parameter set. In this case, a narrow expected output mass range was not known; accordingly, a wide output parameter set of from 20 kDa to 300 kDa was specified. As can be seen in FIG. 2, the deconvolved output from the deconvolution algorithm includes a first peak at 148 kDa and a second peak at 74 kDa. The operator would typically assume that the most intense mass is indicative of a mass of an analyte in the sample. As 148 kDa is a multiple of 74 kDa an operator may intuitively suspect that the peak at 74 kDa is an artefact. However, in the absence of knowledge of the sample, it is not apparent to the operator whether the 74 kDa peak is an artefact or whether the 74 kDa peak corresponds to a mass of an analyte (the sample may contain two analytes, one with a mass of 148 kDa and one with a mass of 74 kDa, but the operator does not know if this is the case or not). In this case, the deconvolution algorithm has attributed a cluster of peaks at around 2965 m/z to a mass of 148 kDa and a charge of 50 electrons and to a mass of 74 kDa and a charge of 25 electrons—accordingly, the 74 kDa peak may be an artefact.

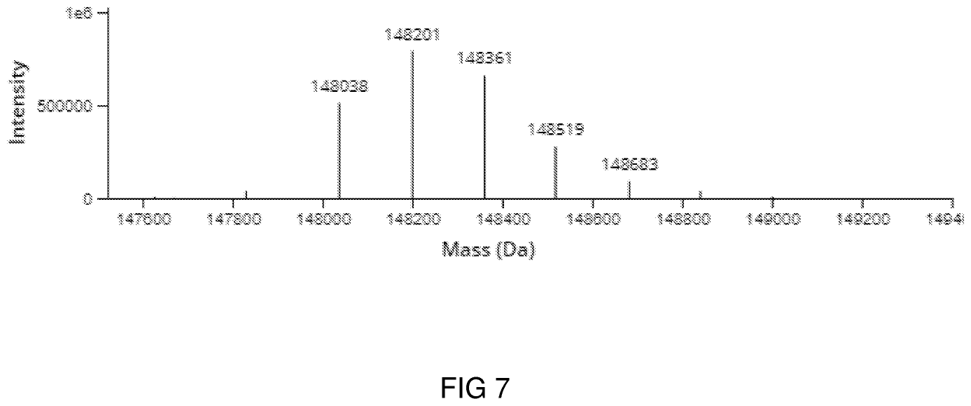

A way around this problem is for an operator to specify as part of the algorithm input a narrow output parameter set including a narrow expected output mass range based on knowledge of the sample analysed. For example, an operator might specify a narrow output parameter set including an expected output mass range of from 100 kDa to 200 kDa based on knowledge of the sample. In such a case, a deconvolution algorithm might then provide a deconvolved output having only a mass peak around 150 kDa (see FIG. 7). However, it is only possible to specify a narrow output parameter set based on knowledge of the sample if the operator has sufficient knowledge of the sample, including expected mass ranges. Further, the expected output mass range must be known in sufficient detail to exclude the mass range of artefacts. As will be appreciated, in the analysis of novel samples, a sufficiently narrow expected output mass range, and therefore an output parameter set, may not be known to the operator. It is therefore not always possible for an operator to specify an appropriate narrow output parameter set based on knowledge of the sample. Further, the operator's perceived knowledge of the sample may be wrong, in which case masses corresponding to analytes may be inadvertently excluded whilst masses corresponding to artefacts may be inadvertently included.

It is a realisation of the present disclosure that relying on operator knowledge of samples may lead to misidentification of artefacts in deconvolved outputs.

It is a further realisation of the present disclosure that operators may use wide output parameter sets; consequently, operators may analyse deconvolution outputs containing artefacts, perhaps wrongly attributing artefacts as being indicative of masses, in turn, when this error is eventually uncovered perceived reliability of the deconvolution algorithm used may be reduced.

It is a further realisation of the present disclosure that automation of the identification of artefacts in deconvolved outputs would both save the time of skilled operators and increase the perceived reliability of the deconvolution algorithm.

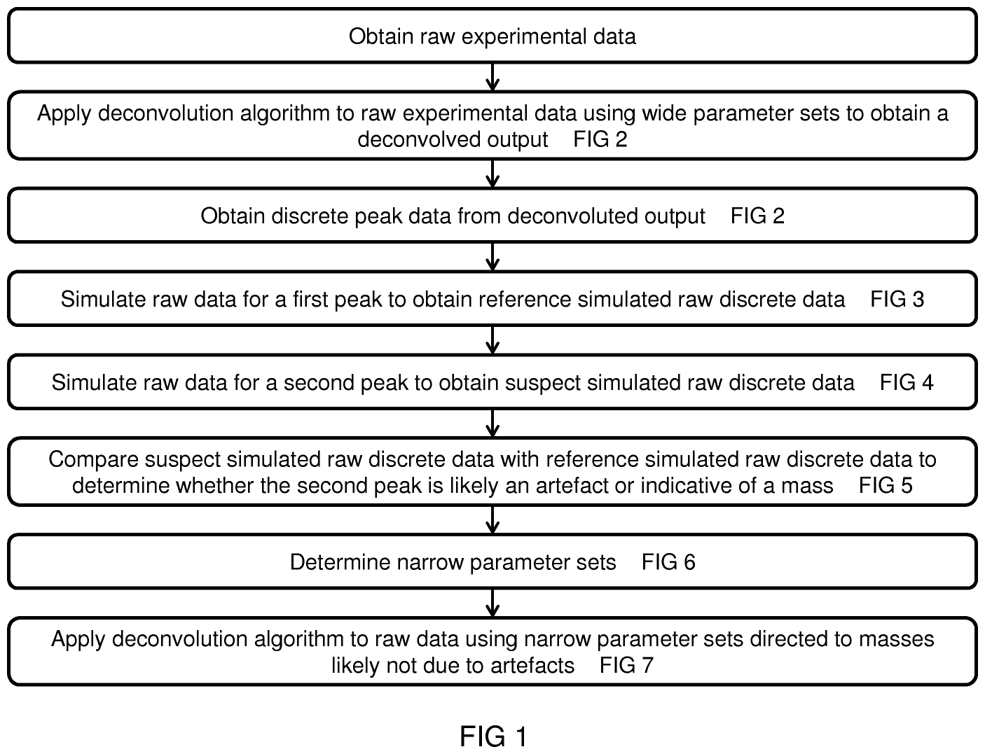

With reference to FIG. 1, the disclosure provides a method of analysis of mass spectrometry data. The method comprises obtaining raw experimental mass spectrometry data.

A first deconvolution of the raw experimental mass spectrometry data is performed using a deconvolution algorithm, a wide first input parameter set, and a wide first output parameter set to obtain a deconvolved output. FIG. 2 shows an example first deconvolved output of masses against intensity.

The deconvolution algorithm may be any known deconvolution algorithm, for example Maximum entropy based deconvolution (MaxEnt1) or Nested Sampling based deconvolution (BayesSpray). The BayesSpray deconvolution algorithm is described in U.S. Pat. No. 8,604,421. The choice of deconvolution algorithm may be made based on the usual factors as is known, for example, molecule size, resolution of instrument used, retention time, peak size, peak shape, required processing speed etc. The wide first input parameter set would typically include the full experimental raw data. In order to make the process efficient, the maximum number of iterations performed by the algorithm may be limited. If the algorithm used requires a peak width or resolution value, then that may be obtained from the raw experimental data automatically.

In this document, the term “wide” applied to a setting or parameter may be understood to mean a value assigned to the setting or parameter that is compromised (or non ideal). This compromise may, for example, comprise assigning an input or output mass to charge or mass range that is larger than actually required by the (a priori unknown) components present in the data. It could additionally or alternatively comprise reducing the number of iterations of the algorithm to improve processing speed, the number of points on the output mass axis, and/or the number of objects used in a nested sampling approach. It could comprise setting a compromise peak width value when the peak width is unknown, and/or multiple different peak widths are present or might be present in the data. It will be appreciated that many other reasons may exist for using a compromised value for a parameter.

Discrete peak data may be obtained from the deconvolved output. The masses within the first deconvolved spectrum can be evaluated as discrete peak data. If the algorithm used to produce the first deconvolved output produces continuum data—such as MaxEnt1—peaks are detected in the first deconvolved output. The widely accepted method of centering the spectrum may be used. This produces discrete data and the centered spectrum is sometimes referred to as a ‘stick plot’, or a ‘centroided spectrum’. If an algorithm which produces discrete data is used—such as BayesSpray— then centering the spectrum is not necessary.

Discrete data produced by centering continuum data may result in a centroided spectrum containing many masses with low intensities. Depending on the algorithm used, the complexity of the raw experimental data and the exact first deconvolution method settings used, these may have been derived from noise in the raw experimental data. Additionally, many users of deconvolution algorithms do not investigate very low intensity masses in a deconvolved spectrum. For these reasons, an intensity threshold may be used to filter the masses in the discrete peak data before further evaluation. For example, an absolute intensity threshold can be used. Alternatively, the largest intensity in the centroided spectrum can be found, and a percentage threshold applied to this in order to calculate an absolute intensity threshold.

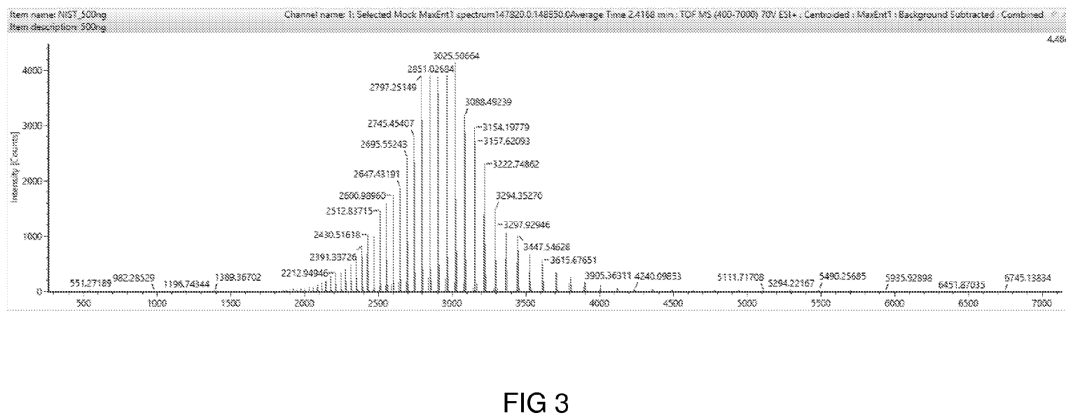

Raw data for a first peak of the discrete peak data is simulated to obtain reference simulated raw discrete data. FIG. 3 shows reference simulated raw discrete data for the peak at 148 kDA in FIG. 2.

In other embodiments, as described below, the simulated data need not be discrete. In such a case, continuum simulated data is compared. Any measure of overlap or correlation of the simulated continuum datasets (reference and suspect) may be used to assess similarity. Such measures may be described as co-efficients of overlap.

The first peak of the discrete peak data may be the most intense peak of the discrete peak data. This is because the most intense mass can generally be assumed to be indicative of a mass of an analyte in the sample. However, if, for example, properties of the sample are known, such information may be used to select the first peak.

Obtaining reference simulated raw discrete data may involve producing simulated (or “mock”) raw continuum data, and then centering this data to obtain the reference simulated raw discrete data. The simulated data may be produced using centroided or stick data, or it may be produced using continuum data in the neighbourhood of the detected peak position in the deconvolved data

Alternatively, the reference simulated discrete data may be produced directly from centroided or stick data, using charge state distribution information if available.

Some deconvolution algorithms infer charge state distributions for each detected peak or each point of the deconvolved mass axis. This charge state distribution information may comprise a proportion of the signal corresponding to each allowed charge state, and/or a minimum and maximum observed charge state, and/or an average charge state, and/or one or more moments or quantiles of the charge state distribution. It will be understood that other methods of summarizing the charge state distribution may be used. It may be preferred that inferred charge state distribution information is utilized in the production of the simulated data. Charge state distributions may be utilized to advantage. For example, the simulated intensity at a given charge state may be very low meaning that data can be ignored (or that a low co-efficient of overlap is determined). In this way, the relative intensities of the simulated data can be compared. In particular, the relative intensities of the simulated data can be compared by calculating the co-efficient of overlap.

The reference simulated raw discrete data may be a list of pairs of m/z and intensity values. The masses may be ordered by increasing mass difference from the reference mass.

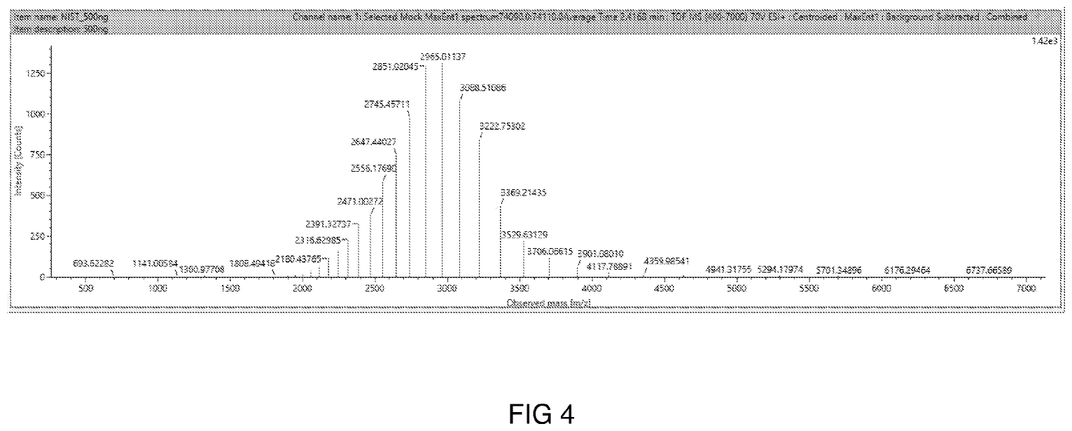

Raw data for a second peak of the discrete peak data is simulated to obtain suspect simulated raw discrete data. FIG. 4 shows suspect simulated raw discrete data for the peak at 74 kDA in FIG. 2.

The second peak of the discrete peak data may be the closest mass to the first peak of the discrete peak data. In other words, once the peak assumed to be indicative of a mass has been selected, the next peak to be analysed may be that closest in mass. The second peak may be referred to as a suspect mass.

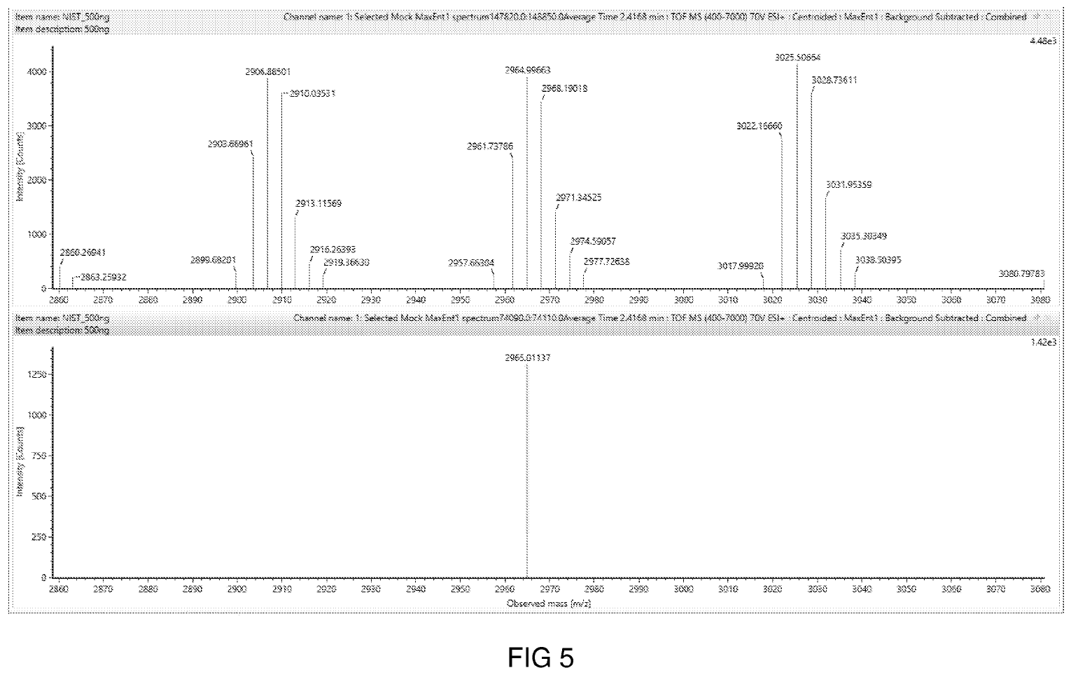

It is then determined whether the second peak is likely an artefact or indicative of a mass by comparing the suspect simulated raw discrete data with the reference simulated raw discrete data. In this example FIG. 5 shows the reference simulated raw discrete data of FIG. 3 (top) adjacent the suspect simulated raw discrete data of FIG. 4 (bottom).

Comparing the suspect simulated raw discrete data with the reference simulated raw discrete data may comprise comparing the m/z values of the suspect simulated raw discrete data with the m/z values of the reference simulated raw discrete data.

Alternatively, it is determined whether the second peak is likely an artefact or indicative of a mass by comparing the suspect simulated raw continuum data with the reference simulated raw continuum data. This may be done by determining a co-efficient of overlap between the suspect simulated raw data and the reference simulated raw data and determining whether the second peak is likely an artefact or indicative of a mass by comparing the co-efficient of overlap to a predetermined threshold.

Comparing the suspect simulated raw continuum data with the reference simulated raw continuum data may comprise comparing the m/z values of the suspect simulated raw continuum data with the m/z values of the reference simulated raw continuum data.

The determining a co-efficient of overlap between the suspect simulated raw data and the reference simulated raw data may comprise determining the co-efficient of overlap for a peak or collection of peaks in the suspect simulated raw data with a peak or collection of peaks in the reference simulated raw data. In other words, it may be decided to compare only a single peak or a collection of peaks and the data processed in an analogous way to the processing of discrete data as described herein.

The determining a co-efficient of overlap between the suspect simulated raw data and the reference simulated raw data may comprise determining the co-efficient of overlap for all or substantially all of the suspect simulated raw data and of the reference simulated raw data. In other words, it may be decided to compare whole simulated raw data sets.

The second peak may be identified as likely indicative of a mass if the suspect simulated raw continuum data does not significantly overlap with the reference simulated raw continuum data. Calculation of a degree of overlap could for example include proportion of baseline overlap, overlap within a fraction of the peak width, relative entropy (Kullback Leibler divergence) or any quantity (probabilistic or otherwise) utilising m/z values and/or intensities that is indicative of whether the two peaks are likely to originate from a single underlying feature.

The second peak may be identified as likely indicative of a mass if an m/z value of the suspect simulated raw discrete data is not sufficiently close to the m/z values of the reference simulated raw discrete data (within an error margin or tolerance e.g. as described herein). In particular, m/z values which are unique—meaning that they are not present (within an error margin or tolerance e.g. as described herein) above some absolute or relative threshold in the reference simulated raw discrete data—may be searched for. A relative threshold may be calculated from intensity values in the reference simulated data, the suspect simulated data, or any other simulated raw or original raw data. A relative threshold may be a proportion of the intensity of the peak or individual datapoints in the reference simulated raw data. Additionally or alternatively, “not present” might mean not present above some fraction or multiple of the intensity of the suspect data intensity.

Once the second peak is identified as likely indicative of a mass, comparing the suspect simulated raw discrete data with the reference simulated raw discrete data may be ceased.

Once the second peak is identified as likely indicative of a mass, the suspect simulated raw discrete data may be added to the reference simulated raw discrete data. As the suspect mass may be considered to be real.

When comparing an m/z in the suspect simulated raw discrete data to an m/z in the reference simulated raw discrete data, we may use the predicted width of the isotope distribution at the charge state (z) of the m/z being considered to decide if the m/z in the suspect simulated raw data is present in the reference simulated raw data. Alternatively, where non-discrete (continuum) simulated data is directly as mentioned above such an additional step may not be necessary as such continuum simulated data may already include the predicted width of the isotope distribution.

The comparing the m/z values of the suspect simulated raw discrete data with the m/z values of the reference simulated raw discrete data may comprise calculating the width of the theoretical isotope distribution at the charge state z of the m/z value under consideration.

The second peak may be identified as likely an artefact if all of the m/z values of the suspect simulated raw discrete data are sufficiently close to the m/z values of the reference simulated raw discrete data (within an error margin or tolerance e.g. as described herein).

For example, with reference to FIG. 5, suppose an m/z of 2965.0114 in the suspect simulated raw discrete data of the suspect mass at 74100 Da is being considered. The reference simulated raw discrete data may contain an m/z 2964.9966 m/z belonging to a real mass of 148200 Da. It may be calculated that the theoretical (or predicted) isotope distribution means that m/z values within 0.45 m/z of 2964.9966 m/z are likely to be isotopes of the deconvolved mass 148200. In this case, it might be calculated that 2965.0114 m/z in the suspect simulated raw discrete data is not unique as it is within 0.45 m/z of 2964.9966 m/z.

The method of analysis of mass spectrometry data may further comprise simulating raw data for a further peak of the discrete peak data to obtain further suspect simulated raw discrete data; and further comprise determining whether the further peak is likely an artefact or indicative of a mass by comparing the further suspect simulated raw discrete data with the reference simulated raw discrete data. In other words, the process may be repeated for further peaks of the discrete peak data.

The above method of analysis of further peaks may be repeated at least 2, 4, 6, 8, 16, 32, 64, 128, 256, 512, 1025, 2048, 4096, 8192, 16384 or more times. The method may be repeated to include further peaks until all significant peaks are analysed (e.g. all peaks above a threshold). If all the (significant) masses are evaluated in this way, a list of pairs of real masses and intensities may be produced. Additionally, for each mass in the list of real masses, simulated raw discrete data may already have been produced and retained.

The method of analysis of mass spectrometry data may further comprise determining a narrow second input parameter set based on the analysis of the simulated raw data.

In this document, the term “narrow” applied to a setting or parameter may be understood to mean a value assigned to the setting or parameter that is optimized (or improved). This optimization may, for example, comprise assigning an input or output mass to charge or mass range that excludes known artifacts. It could additionally or alternatively comprise increasing the number of points on the output mass axis and/or the number of objects used in a nested sampling approach. It could comprise setting an optimized peak width value. It will be appreciated that many other parameters may be optimized using the methods described herein.

The method of analysis of mass spectrometry data may further comprise determining a narrow second input parameter set, comprising setting an input spectrum threshold percentage; setting the smallest m/z value in the reference simulated raw discrete data above the input spectrum threshold percentage as a lower bound of the narrow second input parameter set; and/or setting the largest m/z value in the reference simulated raw discrete data above the input spectrum threshold percentage as an upper bound of the narrow second input parameter set.

Determining the narrow second input parameter set may further comprise: if the second peak is determined as likely indicative of a mass and if the smallest m/z value in the suspect simulated raw discrete data above the input spectrum threshold percentage is smaller than the lower bound of the narrow second input parameter set, setting the smallest m/z value in the suspect simulated raw discrete data as the lower bound of the narrow second input parameter set; and/or and if the largest m/z value in the suspect simulated raw discrete data above the input spectrum threshold percentage is greater than the upper bound of the narrow second input parameter set, setting the largest m/z value in the suspect simulated raw discrete data as the upper bound of the narrow second input parameter set.

Determining the narrow second input parameter set may further comprise: if the or a further peak is determined as likely indicative of a mass if the smallest m/z value in the further suspect simulated raw discrete data above the input spectrum threshold percentage is smaller than the lower bound of the narrow second input parameter set, setting the smallest m/z value in the further suspect simulated raw discrete data as the lower bound of the narrow second input parameter set; and/or if the largest m/z value in the further suspect simulated raw discrete data above the input spectrum threshold percentage is greater than the upper bound of the narrow second input parameter set, setting the largest m/z value in the suspect simulated raw discrete data as the upper bound of the narrow second input parameter set.

The input mass range threshold percentage may be used to optimize producing the second narrow input parameter set (e.g. mass range) to include enough data to produce the final deconvolved spectrum. The input spectrum threshold percentage may be set to zero. This means that all data within the raw experimental data which can be deconvolved is processed in the final deconvolution method. Alternatively, a threshold larger than zero may be used so that enough of the raw experimental data to produce a good quality second (e.g. final) deconvolved spectrum.

The above processes may be used to produce an input m/z range which contains most, if not all, of the data needed to produce the real masses in the deconvolved spectrum.

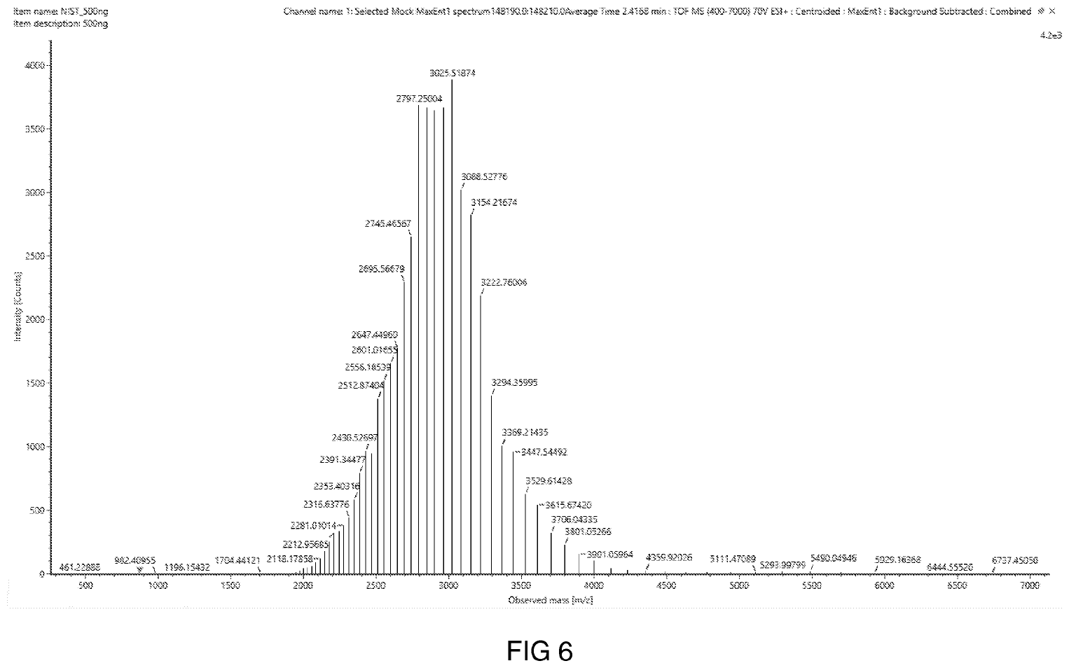

For example, with reference to FIG. 6, which shows the simulated raw discrete data for the most intense likely real mass. The input spectrum threshold percentage may be set to zero. The smallest m/z value (1190) in the reference simulated raw discrete data above the input spectrum threshold percentage may be set as a lower bound of the narrow second input parameter set and the largest m/z value (6450) in the reference simulated raw discrete data above the input spectrum threshold percentage may be set as an upper bound of the narrow second input parameter set. This gives an input m/z range of from 1190 to 6450, in this example.

Alternatively, still with reference to FIG. 6, the input spectrum threshold percentage may be set to 10% giving an input m/z range of from 2310 to 3620, in this example. As a further alternative, the input spectrum threshold percentage may be set to 20% giving an input m/z range of from 2385 to 3455, in this example.

The method of analysis of mass spectrometry data may further comprise determining a narrow second output parameter set, comprising setting an offset value; and setting the smallest of the first peak, the second peak, and/or any further peak(s) determined to be indicative of a mass minus the offset value as a lower bound of the narrow second output parameter set; and/or setting the largest of the first peak, the second peak, and/or any further peak(s) determined to be indicative of a mass plus the offset value as an upper bound of the narrow second output parameter set.

This method may be used when an algorithm allows a single output mass range to be defined, but multiple real masses have been found in the first deconvolved output.

The method of analysis of mass spectrometry data may further comprise determining a narrow second output parameter set, comprising setting an offset value; and setting the first peak, the second peak, and/or any further peak(s) determined to be indicative of a mass plus and minus the offset value as included within the narrow second output parameter set.

This method may be used when an algorithm allows a collection of output mass ranges to be defined and multiple real masses have been found in the first deconvolved output. As will be appreciated, the second output parameter set is a collection of output mass ranges.

As will be appreciated, in this way, the method may further comprise determining a narrow second output parameter set, wherein the narrow parameter set excludes peaks likely to be artefacts and includes peaks indicative of a mass.

It may be important to understand if other related masses are present—for example, masses corresponding to other minor glycoforms of a protein, or masses corresponding to failure sequences in a synthetic peptide. In the simplest case, where there is one real mass, the limited output mass range may be calculated by using an offset value, which may be a percentage of the real mass, so a user can see the presence of absence of closely related masses (only).

The method of analysis of mass spectrometry data may further comprise performing a second deconvolution of the raw experimental mass spectrometry data using a deconvolution algorithm and the narrow second input parameter set determined using a method herein; and/or the narrow second output parameter set determined using a method herein to obtain a second deconvolved output.

As is usual, if the algorithm used requires a peak width or resolution value, then that may be obtained from the raw experimental data automatically.

With the limited input and output parameters defined, the maximum number of iterations performed by the algorithm may be increased and the raw experimental data may be deconvolved to produce a higher quality deconvolved spectrum. The iterations performed by the algorithm may be allowed to proceed until convergence or a large maximum number of iterations has been reached. As will be appreciated, evaluating masses in the first deconvolved spectrum, to determine which are likely real and which are likely artefacts, and only using the likely real masses to calculate a final output mass range can avoid using an unnecessarily wide output mass range for the final deconvolution. Additionally, a more accurate input m/z range to be calculated automatically. This may be advantageous as too wide an output mass range and too wide an input m/z range may lengthen processing time and may result in more artefacts in the deconvolved output.

In the example, the output parameter set may be selected to include the peak at 148 kDa and to exclude the peak at 74 kDa (for example, a mass range of from 140700 Da to 155700 Da).

An example deconvolution output using the narrow second input parameter set and the narrow second output parameter set is shown in FIG. 7. It should be appreciated that using the present method (FIG. 1) the deconvolution output of FIG. 7 has been obtained without an operator having to specify an expected mass range which excludes the artefact (the peak at 74 kDA, FIG. 2) based on knowledge of the sample analysed. Further, the deconvolution output of FIG. 7 excludes artefacts, the peak at 74 kDa in this example. Further still, the deconvolution output of FIG. 7 has been obtained using a method which may be automated.

As will be appreciated, the method of determining whether a peak in a deconvolved output is likely an artefact or indicative of a mass may be used in isolation. Accordingly, there is also provided method of determining whether a second peak in a deconvolved output is likely an artefact or indicative of a mass comprising obtaining discrete peak data from the deconvolved output; simulating raw data for a first peak of the discrete peak data to obtain reference simulated raw discrete data; simulating raw data for the second peak of the discrete peak data to obtain suspect simulated raw discrete data; and determining whether the second peak is likely an artefact or indicative of a mass by comparing the suspect simulated raw discrete data with the reference simulated raw discrete data.

Additionally, there is further provided a method of determining whether a second peak in a deconvolved output is likely an artefact or indicative of a mass comprising: identifying peaks in the deconvolved output; simulating raw data for a first peak of the deconvolved output to obtain reference simulated raw data; simulating raw data for a second peak of the deconvolved output to obtain suspect simulated raw data; determining a co-efficient of overlap between the suspect simulated raw data and the reference simulated raw data; and determining whether the second peak is likely an artefact or indicative of a mass by comparing the co-efficient of overlap to a predetermined threshold.

The methods of determining whether a second peak in a deconvolved output is likely an artefact or indicative of a mass may further comprise one or more or all of the features of the method of analysis of mass spectrometry data described herein.

Once peaks in a deconvolved output have been determined to be either likely an artefact or indicative of a mass, this information may be displayed with the deconvolved output, e.g. peaks of a plot of mass against intensity may be labelled as likely indicative of a mass or likely an artefact.

The whole process as described above may repeated any number of times. For example the result of a second deconvolution of the raw data may be used to produce simulated raw data that can be used to ascertain the reliability of peaks in the second output spectrum. The simulated raw data may alternatively or additionally be used to produce a further refinement of the deconvolution parameters to be used in a third deconvolution step and so on.

A computer program is also provided. The computer program includes instructions which, when the program is executed by a processor, cause the performance of a method described above of analysis of mass spectrometry data.

A further computer program is also provided. The computer program includes instructions which, when the program is executed by a processor, cause the performance of a method described above of determining whether a peak in a deconvolved output is likely an artefact or indicative of a mass.

There is also provided a computer readable medium having instructions stored thereon which, when executed by a processor, cause the performance of a method described above of analysis of mass spectrometry data.

There is also provided a computer readable medium having instructions stored thereon which, when executed by a processor, cause the performance of a method described above of determining whether a peak in a deconvolved output is likely an artefact or indicative of a mass.

There is also provided a system including at least one processor and a computer readable medium, wherein the computer readable medium has instructions stored thereon which, when executed by the at least one processor, cause the system to perform a method described above of analysis of mass spectrometry data.

There is also provided a system including at least one processor and a computer readable medium, wherein the computer readable medium has instructions stored thereon which, when executed by the at least one processor, cause the system to perform a method described above of determining whether a peak in a deconvolved output is likely an artefact or indicative of a mass.

The system may include a processor and a computer readable medium. The computer readable medium may be configured to store instructions for execution by the processor. The processor may include a number of sub-processors which may be configured to work together, e.g. in parallel with each other, to execute the instructions. The sub-processors may be geographically and/or physically separate from each other and may be communicatively coupled to enable coordinated execution of the instructions.

The computer readable medium may be any desired type or combination of volatile and/or non-volatile memory such as, for example, static random access memory (SRAM), dynamic random access memory (DRAM), flash memory, read-only memory (ROM), and/or a mass storage device (including, for example, an optical or magnetic storage device).

The system including the processor and computer readable medium, may be provided in the form of a server, a desktop computer, a laptop computer, or the like.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. A method of analysis of mass spectrometry data comprising:
obtaining raw experimental mass spectrometry data;
performing a first deconvolution of the raw experimental mass spectrometry data using a deconvolution algorithm, a wide first input parameter set, and a wide first output parameter set to obtain a deconvolved output;
obtaining discrete peak data from the deconvolved output;
simulating raw data for a first peak of the discrete peak data to obtain reference simulated raw discrete data;
simulating raw data for a second peak of the discrete peak data to obtain suspect simulated raw discrete data; and
determining whether the second peak is likely an artefact or indicative of a mass by comparing the suspect simulated raw discrete data with the reference simulated raw discrete data.

2. The method of analysis of mass spectrometry data according to claim 1, wherein the first peak of the discrete peak data is the most intense peak of the discrete peak data.

3. The method of analysis of mass spectrometry data according to claim 1, wherein the second peak of the discrete peak data is the closest mass to the first peak of the discrete peak data.

4. The method of analysis of mass spectrometry data according to claim 1, wherein comparing the suspect simulated raw discrete data with the reference simulated raw discrete data comprises comparing the m/z values of the suspect simulated raw discrete data with the m/z values of the reference simulated raw discrete data.

5. The method of analysis of mass spectrometry data according to claim 4, wherein the comparing the m/z values of the suspect simulated raw discrete data with the m/z values of the reference simulated raw discrete data comprises calculating the width of the theoretical isotope distribution at the charge state z of the m/z value under consideration.

6. The method of analysis of mass spectrometry data according to claim 4, wherein the second peak is identified as likely an artefact if all of the m/z values of the suspect simulated raw discrete data are within the m/z values of the reference simulated raw discrete data.

7. The method of analysis of mass spectrometry data according to claim 4, wherein the second peak is identified as likely indicative of a mass if an m/z value of the suspect simulated raw discrete data is not within the m/z values of the reference simulated raw discrete data.

8. The method of analysis of mass spectrometry data according to claim 1, wherein once the second peak is identified as likely indicative of a mass, comparing the suspect simulated raw discrete data with the reference simulated raw discrete data is ceased.

9. The method of analysis of mass spectrometry data according to claim 1, wherein once the second peak is identified as likely indicative of a mass, the suspect simulated raw discrete data is added to the reference simulated raw discrete data.

10. The method of analysis of mass spectrometry data according to claim 1,
further comprising simulating raw data for a further peak of the discrete peak data to obtain further suspect simulated raw discrete data; and
determining whether the further peak is likely an artefact or indicative of a mass by comparing the further suspect simulated raw discrete data with the reference simulated raw discrete data.

11. The method of analysis of mass spectrometry data according to claim 1, further comprising:
determining a narrow second input parameter set, comprising:
setting an input spectrum threshold percentage;
setting the smallest m/z value in the reference simulated raw discrete data above the input spectrum threshold percentage as a lower bound of the narrow second input parameter set; and/or
setting the largest m/z value in the reference simulated raw discrete data above the input spectrum threshold percentage as an upper bound of the narrow second input parameter set.

12. The method of analysis of mass spectrometry data according to claim 11, wherein determining the narrow second input parameter set further comprises:
if the second peak is determined as likely indicative of a mass:
and if the smallest m/z value in the suspect simulated raw discrete data above the input spectrum threshold percentage is smaller than the lower bound of the narrow second input parameter set, setting the smallest m/z value in the suspect simulated raw discrete data as the lower bound of the narrow second input parameter set; and/or
and if the largest m/z value in the suspect simulated raw discrete data above the input spectrum threshold percentage is greater than the upper bound of the narrow second input parameter set, setting the largest m/z value in the suspect simulated raw discrete data as the upper bound of the narrow second input parameter set.

13. The method of analysis of mass spectrometry data according to claim 11, wherein determining the narrow second input parameter set further comprises:
if the or a further peak is determined as likely indicative of a mass:
if the smallest m/z value in the further suspect simulated raw discrete data above the input spectrum threshold percentage is smaller than the lower bound of the narrow second input parameter set, setting the smallest m/z value in the further suspect simulated raw discrete data as the lower bound of the narrow second input parameter set; and/or
if the largest m/z value in the further suspect simulated raw discrete data above the input spectrum threshold percentage is greater than the upper bound of the narrow second input parameter set, setting the largest m/z value in the suspect simulated raw discrete data as the upper bound of the narrow second input parameter set.

14. The method of analysis of mass spectrometry data according to claim 11, wherein the input spectrum threshold percentage is set to zero.

15. The method of analysis of mass spectrometry data according to claim 1, further comprising:
determining a narrow second output parameter set, comprising:
setting an offset value; and
setting the smallest of the first peak, the second peak, and/or any further peak(s) determined to be indicative of a mass minus the offset value as a lower bound of the narrow second output parameter set; and/or
setting the largest of the first peak, the second peak, and/or any further peak(s) determined to be indicative of a mass plus the offset value as an upper bound of the narrow second output parameter set.

16. The method of analysis of mass spectrometry data according to claim 1, further comprising:
determining a narrow second output parameter set, comprising:
setting an offset value; and
setting the first peak, the second peak, and/or any further peak(s) determined to be indicative of a mass plus and minus the offset value as included within the narrow second output parameter set.

17. The method of analysis of mass spectrometry data according to claim 15, further comprising:
performing a second deconvolution of the raw experimental mass spectrometry data using a deconvolution algorithm and the narrow second input parameter set of any of claims 11 to 14; and/or the narrow second output parameter set of claim 15 or 16 to obtain a second deconvolved output.

18. (canceled)

19. A method of analysis of mass spectrometry data comprising:
obtaining raw experimental mass spectrometry data;
performing a first deconvolution of the raw experimental mass spectrometry data using a deconvolution algorithm, a wide first input parameter set, and a wide first output parameter set to obtain a deconvolved output;
identifying peaks in the deconvolved output;
simulating raw data for a first peak of the deconvolved output to obtain reference simulated raw data;
simulating raw data for a second peak of the deconvolved output to obtain suspect simulated raw data;
determining a co-efficient of overlap between the suspect simulated raw data and the reference simulated raw data; and
determining whether the second peak is likely an artefact or indicative of a mass by comparing the co-efficient of overlap to a predetermined threshold.

20. The method of analysis of mass spectrometry data according to claim 19, wherein the determining a co-efficient of overlap between the suspect simulated raw data and the reference simulated raw data comprises determining the co-efficient of overlap for a peak or collection of peaks in the suspect simulated raw data with a peak or collection of peaks in the reference simulated raw data.

21. The method of analysis of mass spectrometry data according to claim 19, wherein the determining a co-efficient of overlap between the suspect simulated raw data and the reference simulated raw data comprises determining the co-efficient of overlap for all or substantially all of the suspect simulated raw data and of the reference simulated raw data.

22. (canceled)

23. (canceled)

24. (canceled)

25. (canceled)
